(19) United States
(12) Patent Application Publication
(10) Pub. No.: US 2020/0283721 A1
(43) Pub. Date: Sep. 10, 2020

(54) CELL CULTURE APPARATUS

(71) Applicant: OLYMPUS CORPORATION, Tokyo (JP)

(72) Inventors: Tatsuya MINAMI, Kanagawa (JP); Yusuke MATSUMOTO, Tokyo (JP)

(73) Assignee: OLYMPUS CORPORATION, Tokyo (JP)

(21) Appl. No.: 16/803,345

(22) Filed: Feb. 27, 2020

(30) Foreign Application Priority Data
Mar. 6, 2019 (JP) 2019-040125

(51) Int. Cl. C12M 1/36; C12M 1/00; C12M 1/34; C12M 1/26; C12M 1/02

(52) U.S. Cl. CPC C12M 41/48; C12M 29/00; C12M 27/00; C12M 33/00; C12M 41/36

(57) ABSTRACT

A cell culture apparatus includes: a supply unit that supplies a culture medium to a culture tank accommodating the culture medium and cells; a discharge unit that discharges the culture medium and the cells from the culture tank; and one or more processors comprising hardware, the one or more processors being configured to determine whether or not cell density in the culture medium in the culture tank has exceeded a preliminarily set predetermined threshold, wherein in response to determining that the cell density has exceeded the predetermined threshold, the one or more processors are configured to cause the discharge unit to discharge a portion of the culture medium and the cells from the culture tank and cause the supply unit to supply a new culture medium to the culture tank to reduce the cell density to the predetermined threshold or less.

### CROSS-REFERENCE TO RELATED APPLICATIONS

This application claims the benefit of Japanese Patent Application No. 2019-040125, the content of which is incorporated herein by reference.

### TECHNICAL FIELD

The present invention relates to a cell culture apparatus.

### BACKGROUND ART

In recent years, with the progress in stem cell research and regenerative medicine and the development of biopharmaceuticals, such as antibody drugs, culture of a large number of cells is required. In culturing a large number of cells, it has become more common to use culture tanks, such as bioreactors, instead of flasks or laboratory dishes (for example, see Patent Literature 1).

Cells in culture may be adversely influenced by stimulation caused by contact with other cells. Hence, it is desirable to optimize the cell density in a culture medium during culture. In a conventional float culture system using a culture tank, such as a bioreactor, to maintain the optimum cell density in the culture medium, the culture medium is changed or culture is finished each time a predetermined cell density is reached.

### CITATION LIST

### Patent Literature

### {PTL 1} Japanese Unexamined Patent Application Publication No. 2011-188777

### SUMMARY OF INVENTION

An aspect of the present invention is a cell culture apparatus including: a supply unit that supplies a culture medium to a culture tank accommodating the culture medium and cells; a discharge unit that discharges the culture medium and the cells from the culture tank; and one or more processors comprising hardware, the one or more processors being configured to determine whether or not cell density in the culture medium in the culture tank has exceeded a preliminarily set predetermined threshold, wherein in response to determining that the cell density has exceeded the predetermined threshold, the one or more processors are configured to cause the discharge unit to discharge a portion of the culture medium and the cells from the culture tank and cause the supply unit to supply a new culture medium to the culture tank to reduce the cell density to the predetermined threshold or less.

### BRIEF DESCRIPTION OF DRAWINGS

FIG. 1 schematically shows the configuration of a cell culture apparatus according to an embodiment of the present invention.

FIG. 2 schematically shows the configuration of a supply unit in FIG. 1.

FIG. 3 schematically shows the configuration of a discharge unit in FIG. 1.

FIG. 4 schematically shows the configuration of a cell culture apparatus according to a modification of the embodiment of the present invention.

FIG. 5 schematically shows the configuration of a supply unit according to a second modification of the embodiment of the present invention.

FIG. 6 schematically shows the configuration of a supply unit according to a third modification of the embodiment of the present invention.

FIG. 7 schematically shows the configuration of another supply unit according to a third modification of the embodiment of the present invention.

FIG. 8 schematically shows the configuration of another discharge unit according to a fifth modification of the embodiment of the present invention.

### DESCRIPTION OF EMBODIMENTS

A cell culture apparatus according to an embodiment of the present invention will be described below with reference to the drawings.

A cell culture apparatus 1 according to this embodiment includes a supply unit 5 that supplies a culture medium W to a culture tank 3 capable of storing the culture medium W and cells S, a discharge unit 7 that discharges the culture medium W and the cells S from the culture tank 3, an observation unit 9 that measures the cell density in the culture medium W in the culture tank 3, an agitation mechanism 11 that agitates the culture medium W in the culture tank 3, and a control unit 13 that controls the supply unit 5, the discharge unit 7, and the observation unit 9.

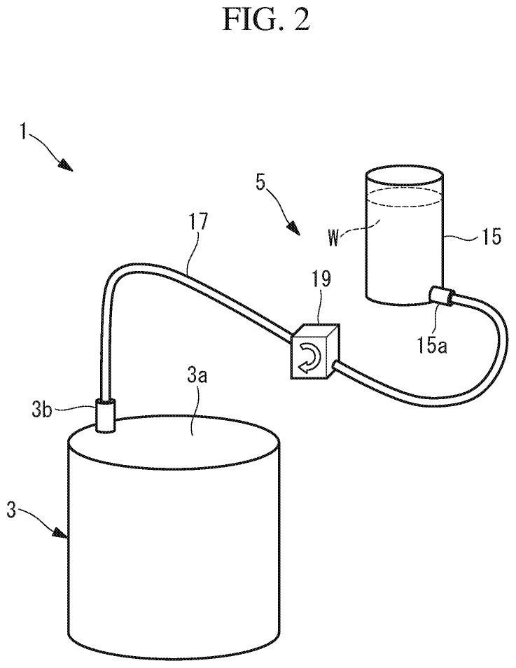

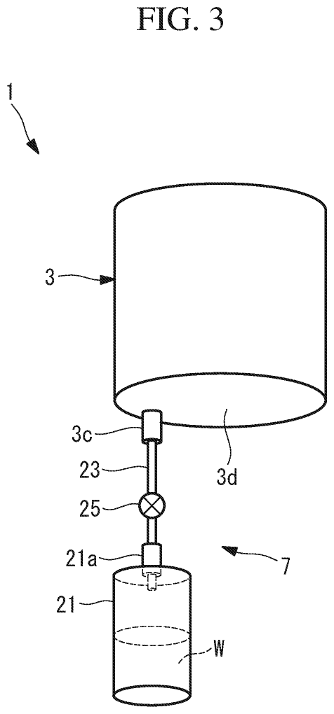

The culture tank 3 is, for example, a bottomed tubular container with a closed top surface 3a. The culture tank 3 is made of an optically transparent material. As shown in, for example, FIG. 2, the culture tank 3 has, in the top surface 3a, a supply port 3b to which the supply unit 5 is connected. Furthermore, as shown in, for example, FIG. 3, the culture tank 3 has, in a bottom surface 3d, a discharge port 3c to which the discharge unit 7 is connected.

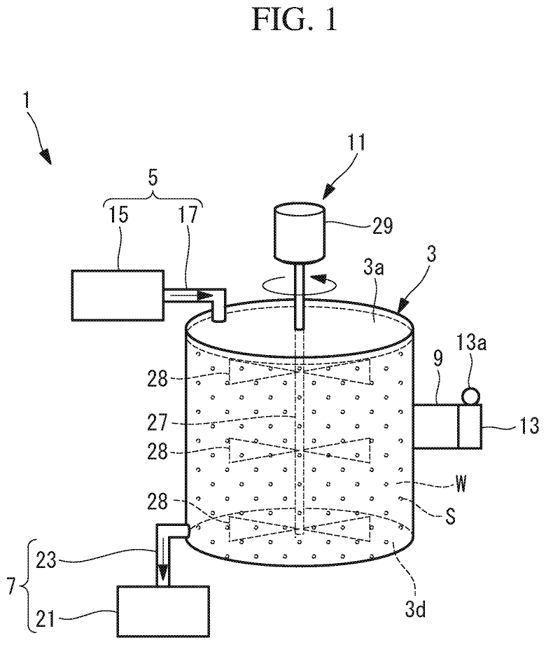

As shown in, for example, FIGS. 1 and 2, the supply unit 5 includes a culture-medium holding container 15 for holding the culture medium W, and a tubular member (supply tubular member) 17, such as a tube, connecting the culture-medium holding container 15 and the culture tank 3 to each other. In FIG. 2, the discharge unit 7 is omitted.

The culture-medium holding container 15 is, for example, a bottomed tubular container. The culture-medium holding container 15 has a discharge port 15a, through which the culture medium W is discharged, near the bottom surface.

The tubular member 17 is connected to the discharge port 15a in the culture-medium holding container 15 at one end thereof in the longitudinal direction and to the supply port 3b in the culture tank 3 at the other end thereof in the longitudinal direction, thus forming a flow path through which the culture medium W is supplied from the culture-medium holding container 15 to the culture tank 3. A liquid feed pump (supply pump) 19, such as a peristaltic pump, for switching between supply and non-supply of the culture medium W from the culture-medium holding container 15 to the culture tank 3 is provided in the tubular member 17.

The liquid feed pump 19 has a receiving unit (not shown) that receives signals from the control unit 13. When the receiving unit receives a signal from the control unit 13, the liquid feed pump 19 is switched between ON and OFF. When switched to an ON state, the liquid feed pump 19 supplies the culture medium W in the culture-medium holding container 15 to the culture tank 3 through the tubular member 17, and, when switched to an OFF state, the liquid feed pump 19 stops the supply of the culture medium W from the culture-medium holding container 15 to the culture tank 3.

As shown in, for example, FIGS. 1 and 3, the discharge unit 7 includes a culture-medium collecting container 21 into which the culture medium W and the cells S are collected, and a tubular member (discharging tubular member) 23, such as a tube, connecting the culture-medium collecting container 21 and the culture tank 3 to each other. In FIG. 3, the supply unit 5 is omitted.

The culture-medium collecting container 21 is, for example, a bottomed tubular container with a closed top surface and has, in the top surface thereof, an inlet 21a, through which the cells S, together with the culture medium W, flow. The culture-medium collecting container 21 is disposed below the culture tank 3 in the direction of gravity.

The tubular member 23 is connected to the discharge port 3c in the culture tank 3 at one end thereof in the longitudinal direction and to the inlet 21a in the culture-medium collecting container 21 at the other end thereof in the longitudinal direction, thus forming a flow path through which the cells S, together with the culture medium W, are discharged from the culture tank 3 to the culture-medium collecting container 21. A gate opening/closing part (discharging valve) 25, such as a valve for switching between discharging and non-discharging of the culture medium W and the cells S from the culture tank 3 to the culture-medium collecting container 21, is provided in the tubular member 23.

The gate opening/closing part 25 has a receiving unit (not shown) that receives a signal from the control unit 13. When the receiving unit receives a signal from the control unit 13, the gate opening/closing part 25 is switched between open and closed. When switched to an open state, the gate opening/closing part 25 opens the flow path of the tubular member 23, allowing the culture medium W and cells S in the culture tank 3 to be discharged to the culture-medium collecting container 21 through the tubular member 23 due to gravity. When switched to an OFF state, the gate opening/closing part 25 closes the flow path of the tubular member 23, thus stopping the discharging of the culture medium W and the cells S from the culture tank 3 to the culture-medium collecting container 21.

The observation unit 9 may use an existing technique for measuring the cell density in the culture medium, and, for example, a configuration including a retroreflector member having an array of a plurality of micro reflective elements may be used. Although illustration thereof is omitted, the observation unit 9 includes, for example: an objective lens disposed on a side of the culture tank 3; an illumination optical system that radiates illumination light onto the culture medium W in the culture tank 3 from outside the culture tank 3, through the objective lens; a retroreflector member that reflects the illumination light passing through the culture tank 3 back to the culture tank 3 via the same path as the incident path; an image capturing unit that captures an image of the illumination light transmitted again through the culture medium W in the culture tank 3 and collected by the objective lens; and an image analysis unit that calculates the cell density in the culture medium W in the culture tank 3 on the basis of the image captured by the image capturing unit.

In the observation unit 9, when the illumination light is radiated from the illumination optical system onto the culture medium W in the culture tank 3, the illumination light passing through the culture medium W is reflected by the retroreflector member and returns to the culture tank 3 via the same path as the path along which the illumination light was incident on the retroreflector member. Then, the illumination light returned to the culture tank 3 is transmitted through the culture medium W in the culture tank 3 again, the illumination light is collected by the objective lens, and an image thereof is captured by the image capturing unit. Then, the image analysis unit calculates the cell density in the culture medium W in the culture tank 3 on the basis of the number of the cells S in the image captured by the image capturing unit.

The agitation mechanism 11 includes, for example, an agitation shaft 27 inserted into the culture tank 3 through the top surface 3a of the culture tank 3, a plurality of agitation blades 28 provided on the agitation shaft 27, and a motor 29 that axially rotates the agitation shaft 27.

In the agitation mechanism 11, when the motor 29 axially rotates the agitation shaft 27, the agitation shaft 27 and the agitation blades 28 rotate in the culture medium W, agitating the culture medium W in the culture tank 3. By agitating the culture medium W with the agitation mechanism 11, it is possible to allow the cells S to float in the culture medium W.

Although illustration thereof is omitted, the control unit 13 includes, for example: a timer; a storage unit, such as a hard disk drive; a CPU (central processing unit); and a RAM (random access memory). The control unit 13 may be, for example, a PC (personal computer). Furthermore, as shown in FIG. 1, the control unit 13 includes a transmitting unit 13a that transmits signals to the supply unit 5 and the discharge unit 7. In the control unit 13, the CPU executes the control program stored in the memory to implement the following functions.

For example, the control unit 13 periodically actuates the observation unit 9 to sequentially measure the cell density in the culture medium W in the culture tank 3. The control unit 13 also transmits driving signals to the discharge unit 7 and the supply unit 5 via the transmitting unit 13a when the cell density measured by the observation unit 9 has reached a predetermined threshold. Then, the control unit 13 reduces the cell density in the culture medium W in the culture tank 3 to the predetermined threshold or less by causing the discharge unit 7 to discharge a portion of the culture medium W and the cells S from the culture tank 3 and causing the supply unit 5 to supply a new culture medium W to the culture tank 3.

The control unit 13 may be disposed adjacent to the observation unit 9 or may be disposed away from the observation unit 9. The control unit 13 may simultaneously transmit the driving signals to both of the discharge unit 7 and the supply unit 5 or may transmit the driving signals to the discharge unit 7 and the supply unit 5 with a time lag. For example, the control unit 13 may transmit the driving signal to the supply unit 5 after a certain time has elapsed from the transmission of the driving signal to the discharge unit 7. Furthermore, for example, the control unit 13 may simultaneously transmit the driving signals to both of the discharge unit 7 and the supply unit 5, the discharge unit 7 may be actuated upon receipt of the driving signal, and the supply unit 5 may be actuated after a certain time has elapsed from the receipt of the driving signal.

The control unit 13 may stop the operations of the discharge unit 7 and the supply unit 5 by transmitting stop signals to the discharge unit 7 and the supply unit 5 after a certain time has elapsed from the actuation of the discharge unit 7 and the supply unit 5. Alternatively, the operations of the discharge unit 7 and the supply unit 5 may be automatically stopped after a certain time has elapsed from the actuation thereof. Alternatively, the control unit 13 may stop the discharge unit 7 and the supply unit 5 when the cell density in the culture medium W in the culture tank 3, detected by the observation unit 9, has dropped to the predetermined threshold or less.

The control unit 13 may transmit the signals to the discharge unit 7 and the supply unit 5 either via wires or wirelessly.

Next, the effects of the cell culture apparatus 1 according to this embodiment will be described.

When the cells S are cultured by using the cell culture apparatus 1 having the above-described configuration, the culture medium W and the cells S are accommodated in the culture tank 3, and the culture medium W in the culture tank 3 is agitated with the agitation mechanism 11 to allow the cells S to float in the culture medium W.

Next, in a state in which the cells S are cultured in the culture tank 3, the control unit 13 controls the observation unit 9 such that the observation unit 9 sequentially measures the cell density in the culture medium W in the culture tank 3. When the cell density measured by the observation unit 9 exceeds a predetermined threshold, the control unit 13 transmits driving signals to the discharge unit 7 and the supply unit 5.

The driving signal from the control unit 13 serves as a trigger and switches the gate opening/closing part 25 of the discharge unit 7 to an open state. As a result, a portion of the culture medium W and cells S in the culture tank 3 is discharged to the culture-medium collecting container 21 through the tubular member 23. The driving signal from the control unit 13 serves as a trigger and switches the liquid feed pump 19 of the supply unit 5 to an ON state. As a result, a new culture medium W is supplied from the culture-medium holding container 15 to the culture tank 3 through the tubular member 17. This way, the cell density in the culture medium W in the culture tank 3 is reduced to the predetermined threshold or less.

When the cell density in the culture medium W in the culture tank 3 has dropped to the predetermined threshold or less, discharging of the culture medium W and the cells S from the culture tank 3, performed by the discharge unit 7, and supply of a new culture medium W to the culture tank 3, performed by the supply unit 5, are stopped, and culture of the cells S is continued. When the cell density in the culture medium W in the culture tank 3 exceeds the predetermined threshold again, the control unit 13 controls the discharge unit 7 and the supply unit 5 such that the cell density in the culture medium W in the culture tank 3 is reduced to the predetermined threshold or less.

Accordingly, with the cell culture apparatus 1 according to this embodiment, it is possible to easily maintain an optimum cell density in the culture medium W in the culture tank 3 and thus to improve the production efficiency of the cells S. Furthermore, by changing a portion of the culture medium W in the culture tank 3 with a new culture medium W during culture of the cells S, deterioration of the culture medium W in the culture tank 3 can be suppressed.

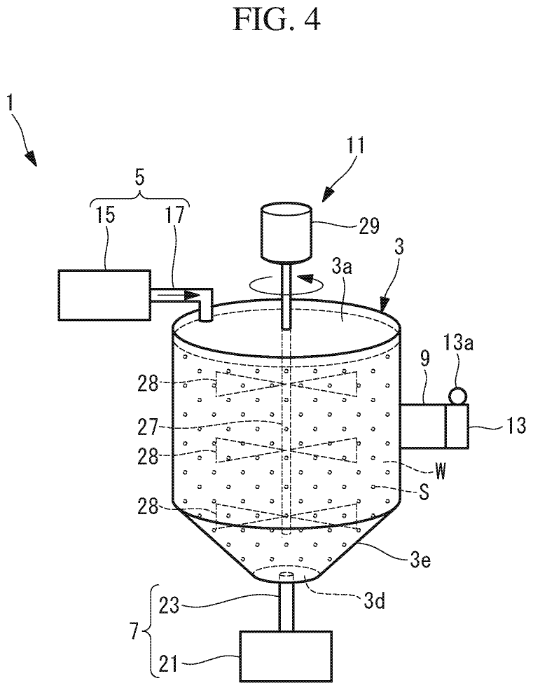

In this embodiment, for example, as shown in FIG. 4, the culture tank 3 may have a tapered portion 3e tapered toward the bottom surface 3d from an intermediate position in the depth direction.

With this configuration, the cells S floating in the lower part of the culture tank 3 move along the inclination of the tapered portion 3e and thus can be easily gathered near the discharge port 3c in the bottom surface 3d. Hence, it is easy to collect the cells S.

This embodiment may be modified as follows.

In a first modification, the control unit 13 may control the driving of the agitation mechanism 11. In this case, the discharge unit 7 may be caused to discharge a portion of the culture medium W and cells S in the culture tank 3 after the control unit 13 has stopped agitation of the culture medium W with the agitation mechanism 11 or has reduced the agitation speed with which the agitation mechanism 11 agitates the culture medium W.

According to this modification, by stopping the agitation of the culture medium W or reducing the agitation speed with the control unit 13 when the culture medium W and cells S in the culture tank 3 are discharged, it is possible to allow the cells S in the culture medium W to be sedimented together near the bottom surface 3d of the culture tank 3 due to their own weight. Thus, compared with a state in which the cells S are scattered in the culture medium W, it is possible to efficiently discharge the cells S from the discharge port 3c provided in the bottom surface 3d of the culture tank 3.

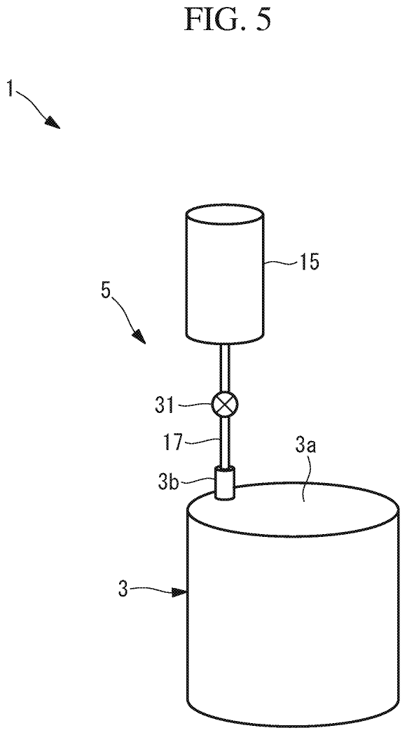

In a second modification, for example, as shown in FIG. 5, the culture-medium holding container 15 may be disposed above the culture tank 3 in the direction of gravity. Then, the culture medium W may be supplied from the culture-medium holding container 15 to the culture tank 3 through the tubular member 17 by means of gravity. The discharge unit 7 is omitted in FIG. 5.

In this case, the tubular member 17 may be provided with a gate opening/closing part (supply valve) 31, such as a valve for opening and closing the flow path, instead of the liquid feed pump 19. Furthermore, the gate opening/closing part 31 may have a receiving unit (not shown) that receives a signal from the control unit 13, and opening and closing thereof may be switched when the receiving unit receives a signal from the control unit 13.

According to this modification, when the gate opening/closing part 31 is switched to an open state, the flow path of the tubular member 17 is opened, and the culture medium W in the culture-medium holding container 15 is supplied to the culture tank 3 through the tubular member 17 due to gravity. In contrast, when the gate opening/closing part 31 is switched to a closed state, the flow path of the tubular member 17 is closed, and the supply of the culture medium W from the culture-medium holding container 15 to the culture tank 3 is stopped.

Accordingly, it is possible to switch between supply and non-supply of the culture medium W to the culture tank 3 with a simple configuration, in which simply the flow path of the tubular member 17 is opened or closed by the gate opening/closing part 31.

In a third modification, the supply unit 5 may have a temperature control means (not shown) for controlling the temperature of the culture medium W in the culture-medium holding container 15.

The temperature control means makes it possible to maintain the culture medium W in the culture-medium holding container 15 at a temperature suitable for culture or at a temperature suitable for preservation.

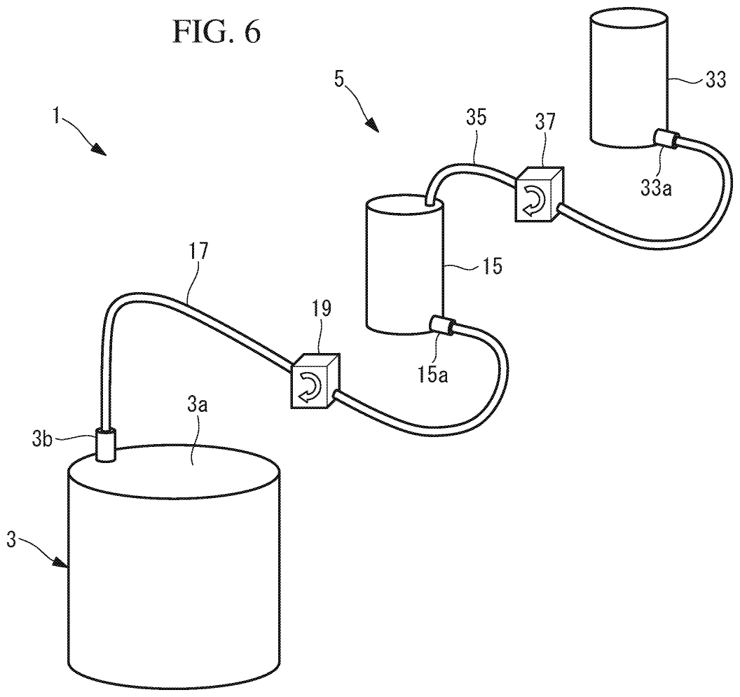

In this modification, as shown in, for example, FIG. 6, the supply unit 5 may further include a culture-medium holding container 33 and a tubular member 35 for supplying the culture medium W to the culture-medium holding container 15. Hereinbelow, the culture-medium holding container 15 and the tubular member 17 may also be referred to as the first culture-medium holding container 15 and the first tubular member 17, and the culture-medium holding container 33 and the tubular member 35 may also be referred to as the second culture-medium holding container 33 and the second tubular member 35. The discharge unit 7 is omitted in FIG. 6.

The second culture-medium holding container 33 and the second tubular member 35 may have the same configuration as the first culture-medium holding container 15 and the first tubular member 17. For example, the second culture-medium holding container 33 may have a discharge port 33a near the bottom surface. Furthermore, the second tubular member 35 may have a liquid feed pump 37, such as a peristaltic pump, for switching between supply and non-supply of the culture medium W from the second culture-medium holding container 33 to the first culture-medium holding container 15.

In this case, by using the temperature control means, the first culture-medium holding container 15 may maintain the culture medium W at a temperature suitable for culturing the cells S, for example, 37° C., and the second culture-medium holding container 33 may maintain the culture medium W at a temperature suitable for preservation, for example, 4° C.

By maintaining the culture medium W at, for example, 4° C. in the second culture-medium holding container 33, deterioration of the culture medium W is prevented. Furthermore, by heating the culture medium W supplied from the second culture-medium holding container 33 to the first culture-medium holding container 15 to, for example, 37° C. in the first culture-medium holding container 15, it is possible to supply the culture medium W at a temperature suitable for culture to the culture tank 3 and, thus, to reduce the stress on the cells S caused by temperature changes.

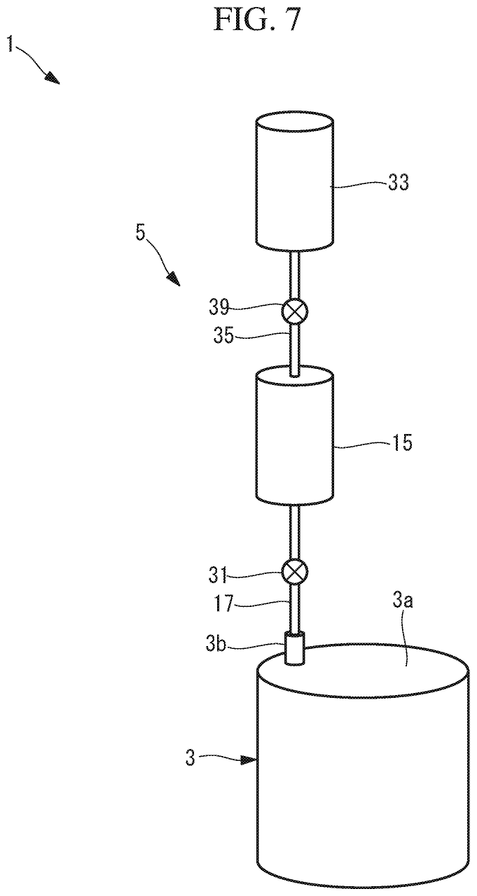

According to this modification, for example, as shown in FIG. 7, the second culture-medium holding container 33 may be disposed above the first culture-medium holding container 15 in the direction of gravity. The culture medium W may be supplied from the second culture-medium holding container 33 to the first culture-medium holding container 15 through the second tubular member 35 by means of gravity. The discharge unit 7 is omitted in FIG. 7.

In this case, the tubular member 17 may be provided with the gate opening/closing part 31, instead of the liquid feed pump 19, and the second tubular member 35 may be provided with a gate opening/closing part 39, such as a valve for opening and closing the flow path, instead of the liquid feed pump 37. The control unit 13 may control opening/closing of the flow path of the first tubular member 17 with the gate opening/closing part 31 and opening/closing of the flow path of the second tubular member 35 with the gate opening/closing part 39.

In a fourth modification, the discharge unit 7 may include a discharging pump (not shown), such as a liquid feed pump, provided in the tubular member 23, instead of the gate opening/closing part 25.

In this case, the culture-medium collecting container 21 does not need to be disposed below the culture tank 3 in the direction of gravity and may be disposed at any desired position.

In this configuration, the discharging pump may be switched ON/OFF, and thus, the discharging of the culture medium W from the culture tank 3 may be controlled, when the receiving unit of the discharging pump receives a signal from the control unit 13.

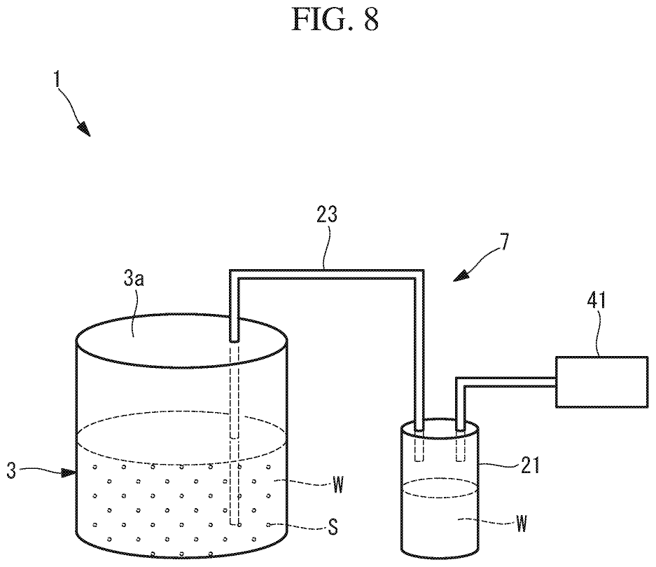

In a fifth modification, as shown in, for example, FIG. 8, the discharge unit 7 may include a negative-pressure supply means, such as a suction pump 41 for applying a negative pressure to the culture-medium collecting container 21, instead of the gate opening/closing part 25. The supply unit 5 is omitted in FIG. 8.

In this case, the suction pump 41 may have a receiving unit (not shown), and the suction pump 41 may be switched ON/OFF when the receiving unit receives a signal from the control unit 13. When the suction pump 41 is switched to an ON state, a negative pressure may be applied to the culture-medium collecting container 21, so that the culture medium W and cells S in the culture tank 3 are sucked into the culture-medium collecting container 21 through the tubular member 23, and, when the suction pump 41 is switched to an OFF state, suction of the culture medium W and cells S in the culture tank 3 may be stopped.

Although the embodiment of the present invention has been described in detail above with reference to the drawings, detailed configurations are not limited to those described in this embodiment, and design changes and the like not departing from the scope of the present invention are also included. For example, the present invention is not limited to one applied to the above-described embodiment and modifications, but may be applied to an embodiment in which the embodiment and the modifications are appropriately combined; it is not specifically limited.

Furthermore, although this embodiment employs a configuration in which the observation unit 9 has the retroreflector member, instead of this, for example, a configuration in which a stereo optical system is provided or a configuration in which an endoscope-observation optical system is provided may be used. A configuration in which the turbidity of the culture medium W is measured may also be used.

In the configuration in which an endoscope-observation optical system is provided, although illustration thereof is omitted, the observation unit 9 includes, for example: an endoscope having an illumination unit that generates illumination light for irradiating the cells S in the culture tank 3, an image-forming optical system that forms an image of observation light coming from the cells S irradiated with the illumination light, an image capturing unit that captures an image of the observation light formed by the image-forming optical system, and a housing that has a transparent part through which the illumination light and the observation light can pass and that accommodates the illumination unit, the image-forming optical system, and the image capturing unit; and an image processing unit that processes the image captured by the image capturing unit.

In this case, the housing is put in the culture medium W in the culture tank 3, and the illumination light is radiated from the illumination unit in the housing onto the cells S floating in the culture medium W through the transparent part in the housing. Then, the image-forming optical system forms an image of the observation light coming from the cells S irradiated with the illumination light and entering the housing through the transparent part, and the image capturing unit captures an optical image of the observation light. Then, the image processing unit estimates the cell density in the culture medium W in the culture tank 3 on the basis of the number of the cells S included in the image captured by the endoscope.

In the configuration in which a stereomeasurement optical system is provided, the observation unit 9 may include, for example: a stereo optical system that forms two images of one cell S floating in the culture medium W, the two images being viewed from different perspectives and thus having parallax; a stereo image-capturing optical system having image capturing units that capture the two images formed by the stereo optical system; an optically transparent housing that accommodates the stereo image-capturing optical system; and an image analysis unit that calculates the cell density in the culture tank 3 on the basis of the plurality of images captured by the stereo image-capturing optical system.

In this case, the housing is put in the culture medium W in the culture tank 3, the stereo optical system in the housing forms two images of one cell S floating in the culture medium W, the two images being viewed from different perspectives and thus having parallax, and the image capturing units capture the thus-formed two images. Then, the image analysis unit identifies the position of the cell S included in the two images captured by the image capturing units, and the cell density in the culture medium W in the culture tank 3 is calculated on the basis of the number of the cells S in a predetermined area.

In the configuration in which the turbidity of the culture medium W in the culture tank 3 is measured, although illustration thereof is omitted, the observation unit 9 may include, for example: a light source that radiates illumination light onto the culture medium W in the culture tank 3; a light-receiving part that receives the illumination light passing through the culture medium W; and an arithmetic processing unit that measures cell density in the culture medium W in the culture tank 3 by using the degree of transmission of the light received by the light-receiving part as an index.

Furthermore, although the cylindrical culture tank 3 formed of an optically transparent material and having a bottom has been described as an example in the above-described embodiment, a culture tank having any desired shape, such as a bag shape, a ball shape, or a box shape may be used. For example, a disposable bag-shaped culture tank may be used. The culture tank may be made of any desired material, such as a hard material or a soft material, such as vinyl. The culture tank 3 does not necessarily have to be transparent overall, and the culture tank 3 may partially have a transparent part through which light, such as the illumination light and the observation light, passes.

Furthermore, although the agitation mechanism according to the above-described embodiment is configured such that the motor 29 axially rotates the agitation shaft 27, it is possible to rotate an agitation member disposed inside the culture tank 3 with a magnetic force from outside the culture tank 3, as in a magnetic stirrer. The culture medium W may be agitated by supplying the culture medium W or a gas from the bottom surface 3d of the culture tank 3, thus producing an upward flow. A mechanism that can cause the cells S to float without applying stress on the cells S as much as possible is preferred.

Although FIGS. 2 and 6 show examples in which the culture-medium holding containers 15 and 33 of the supply unit 5 have the discharge ports 15a and 33a near the bottom surfaces, the discharge ports 15a and 33a may be provided at positions other than the vicinity of the bottom surface. In this case, the tubular member 17 is led out from the discharge port 15a and inserted into the culture tank 3. It is desirable to dispose the opening ends of the tubular members 17 and 35 near the bottom surfaces of the culture-medium holding containers 15 and 33.

In the above-described embodiment, the control unit 13 can optimize the pattern of change in cell density in the culture tank 3 with time by controlling the observation unit 9, the discharge unit 7, and the supply unit 5. For example, when the cell density measured by the observation unit 9 has reached a predetermined threshold, the control unit 13 controls the discharge unit 7 and the supply unit 5 so as to collect a large number of cells. In this case, although the cell density markedly changes with time, the cell collecting frequency can be reduced. For example, when the cell density measured by the observation unit 9 has reached a predetermined threshold, the control unit 13 controls the discharge unit 7 and the supply unit 5 so as to collect a small number of cells. In this case, although the cell collecting frequency increases, the cell density can be maintained near the threshold. The pattern of cell-density change with time can be appropriately optimized according to the type of the cells to be cultured or the culture conditions.

From the above-described embodiment, the following aspect is derived.

An aspect of the present invention is a cell culture apparatus including: a supply unit that supplies a culture medium to a culture tank capable of accommodating the culture medium and cells; a discharge unit that discharges the culture medium and the cells from the culture tank; an observation unit that measures cell density in the culture medium in the culture tank; and a control unit that, when the cell density measured by the observation unit has exceeded a preliminarily set predetermined threshold, causes the discharge unit to discharge a portion of the culture medium and the cells from the culture tank and causes the supply unit to supply a new culture medium to the culture tank to reduce the cell density to the predetermined threshold or less.

According to this aspect, the observation unit measures the cell density in the culture medium in the culture tank in a state in which the cells, together with the culture medium, are accommodated in the culture tank and cultured. When the cell density measured by the observation unit has exceeded the predetermined threshold, the control unit controls the discharge unit so as to discharge a portion of the culture medium and the cells from the culture tank and controls the supply unit so as to supply a new culture medium to the culture tank. By doing so, the cell density in the culture medium in the culture tank is reduced to the predetermined threshold or less. As a result, it is possible to easily maintain optimum cell density in the culture medium and thus to improve cell production efficiency. Furthermore, by changing a portion of the culture medium in the culture tank with a new culture medium during culture of cells, deterioration of the culture medium in the culture tank can be delayed.

According to the above aspect, the supply unit may include a culture-medium holding container that holds the culture medium to be supplied to the culture tank, and a supply tubular member that connects the culture-medium holding container and the culture tank such that the culture medium can pass therethrough, and the supply tubular member may be provided with a supply pump or a supply valve that switches between supply and non-supply of the culture medium from the culture-medium holding container to the culture tank.

With this configuration, the culture medium held in the culture-medium holding container is supplied to the culture tank via the supply tubular member. In this case, it is possible to start or stop the supply of the culture medium from the culture-medium holding container to the culture tank with a simple configuration in which simply the supply pump or the supply valve is actuated.

According to the above aspect, the discharge unit may include a culture-medium collecting container into which the culture medium and cells discharged from the culture tank are collected, and a discharging tubular member that connects the culture tank and the culture-medium collecting container such that the culture medium can pass therethrough, and the discharging tubular member may be provided with a discharging pump or a discharging valve that switches between discharging and non-discharging of the culture medium and the cells from the culture tank to the culture-medium collecting container.

With this configuration, the culture medium and the cells in the culture tank are discharged into the culture-medium collecting container via the discharging tubular member. In this case, it is possible to start or stop discharging of the culture medium and the cells from the culture tank to the culture-medium collecting container with a simple configuration in which simply the discharging pump or the discharging valve is actuated.

According to the above aspect, the discharge unit may include a culture-medium collecting container into which the culture medium and cells discharged from the culture tank are collected, a discharging tubular member that connects the culture tank and the culture-medium collecting container such that the culture medium and the cells can pass therethrough, and a suction pump that applies a negative pressure to the culture-medium collecting container.

With this configuration, the culture medium and the cells in the culture tank are discharged into the culture-medium collecting container via the discharging tubular member. In this case, by applying a negative pressure to the culture-medium collecting container with the suction pump, it is possible to draw the culture medium and the cells in the culture tank into the culture-medium collecting container. Furthermore, by releasing the application of the negative pressure to the culture-medium collecting container with the suction pump, it is possible to stop the drawing of the culture medium and the cells in the culture tank into the culture-medium collecting container. Accordingly, it is possible to start or stop discharging of the culture medium and the cells from the culture tank into the culture-medium collecting container with a simple configuration in which simply the operation of the suction pump is controlled.

According to the above aspect, the cell culture apparatus may further include an agitation mechanism that agitates the culture medium in the culture tank, and the control unit may cause the discharge unit to discharge the culture medium and the cells after stopping the agitation of the culture medium with the agitation mechanism or after reducing a speed at which the culture medium is agitated by the agitation mechanism.

With this configuration, the agitation mechanism agitates the culture medium in the culture tank. In this case, by stopping, with the control unit, the agitation of the culture medium or reducing the agitation speed when the culture medium and the cells in the culture tank are discharged, it is possible to allow the cells in the culture medium to be sedimented by their own weight and, thus, to gather the cells in the culture medium. Thus, compared with a state in which the cells are scattered in the culture medium, it is possible to efficiently discharge the cells.

According to the above aspect, the control unit may output a signal when the cell density measured by the observation unit has exceeded the predetermined threshold, and the supply unit and the discharging unit may be controlled according to the signal outputted from the control unit.

With this configuration, when the cell density in the culture medium has exceeded the predetermined threshold, it is possible to quickly actuate the supply unit and the discharging unit to adjust the cell density.

According to the above aspect, the control unit may transmit/receive the signal to/from the supply unit and the discharge unit via wires or wirelessly.

### REFERENCE SIGNS LIST

1 cell culture apparatus
5 supply unit
7 discharge unit
9 observation unit
11 agitation mechanism
13 control unit
15 first culture-medium holding container (culture-medium holding container)
17 first tubular member (supply tubular member)
19 liquid feed pump (supply pump)
21 culture-medium collecting container
23 tubular member (discharging tubular member)
25 gate opening/closing part (discharging valve)
31 gate opening/closing part (supply valve)
41 suction pump
S cell
W culture medium

1. A cell culture apparatus comprising:
a supply unit that supplies a culture medium to a culture tank accommodating the culture medium and cells;
a discharge unit that discharges the culture medium and the cells from the culture tank; and
one or more processors comprising hardware, the one or more processors being configured to determine whether or not cell density in the culture medium in the culture tank has exceeded a preliminarily set predetermined threshold, wherein
in response to determining that the cell density has exceeded the predetermined threshold, the one or more processors are configured to cause the discharge unit to discharge a portion of the culture medium and the cells from the culture tank and cause the supply unit to supply a new culture medium to the culture tank to reduce the cell density to the predetermined threshold or less.

2. The cell culture apparatus according to claim 1, wherein
the supply unit includes a culture-medium holding container that holds the culture medium to be supplied to the culture tank, and a supply tube that connects the culture-medium holding container and the culture tank such that the culture medium passes therethrough, and
the supply tube is provided with a supply pump or a supply valve that switches between supply and non-supply of the culture medium from the culture-medium holding container to the culture tank.

3. The cell culture apparatus according to claim 1, wherein
the discharge unit includes a culture-medium collecting container into which the culture medium and cells discharged from the culture tank are collected, and a discharging tube that connects the culture tank and the culture-medium collecting container such that the culture medium passes therethrough, and
the discharging tube is provided with a discharging pump or a discharging valve that switches between discharging and non-discharging of the culture medium and the cells from the culture tank to the culture-medium collecting container.

4. The cell culture apparatus according to claim 1, wherein the discharge unit includes a culture-medium collecting container into which the culture medium and cells discharged from the culture tank are collected, a discharging tube that connects the culture tank and the culture-medium collecting container such that the culture medium and the cells pass therethrough, and a suction pump that applies a negative pressure to the culture-medium collecting container.

5. The cell culture apparatus according to claim 1, further comprising an agitator that agitates the culture medium in the culture tank, wherein
the one or more processors are configured to cause the discharge unit to discharge the culture medium and the cells after stopping the agitation of the culture medium with the agitator or after reducing a speed at which the culture medium is agitated by the agitator.

6. The cell culture apparatus according to claim 1, wherein
the one or more processors are configured to output a signal when the cell density has exceeded the predetermined threshold, and
the supply unit and the discharging unit are controlled according to the outputted signal.

7. The cell culture apparatus according to claim 6, wherein the one or more processors are configured to transmit/receive the signal to/from the supply unit and the discharge unit via wires or wirelessly.
